(19) United States
(12) Patent Application Publication
(10) Pub. No.: US 2026/0216558 A1
(43) Pub. Date: Jul. 30, 2026

(54) WEARABLE ASSISTANCE DEVICE

(71) Applicant: Vanderbilt University, Nashville, TN (US)

(72) Inventors: Karl E. Zelik, Nashville, TN (US); Derek Wolf, Nashville, TN (US); Jaime Suzanne Fine, Nashville, TN (US); Chad Ice, Nashville, TN (US); Paul Slaughter, Nashville, TN (US)

(21) Appl. No.: 19/146,083

(22) PCT Filed: Jan. 9, 2024
(86) PCT No.: PCT/US2024/010830

§ 371 Date: Jul. 7, 2025

Related U.S. Application Data
(60) Provisional application No. 63/479,065, filed on Jan. 9, 2023.

(51) Int. Cl. A63B 21/00

(52) U.S. Cl. CPC A63B 21/4025; A63B 21/00181; A63B 21/157; A63B 21/4007

(57) ABSTRACT

A wearable assistance device that includes a lower-body interface, an upper-body interface, and an elastic member coupling the upper-body interface to the lower-body interface. The elastic member has first and second elements associated with the lower-body and upper-body interfaces, respectively. The first and second elements are configured to provide an assistive force to the back of the wearer. A clutch mechanism is associated with the elastic member. The clutch mechanism is configured for selectively adjusting the assistive force provided by the first and second elements of the elastic member. A transmission member is positioned between the first and second elements and extends from the clutch mechanism such that the clutch mechanism is spaced away from the second element of the elastic member. The transmission member is configured to redirect forces between the first and second elements.

### CROSS-REFERENCE TO RELATED APPLICATION

This application claims priority to U.S. Provisional Application Ser. No. 63/479,065, filed on Jan. 9, 2023, and entitled Wearable Assistance Devices and Systems with Flexible or Curved Transmission and Methods of Use, the subject matter of which is herein incorporated by reference in its entirety.

### GOVERNMENT FUNDING

This invention was made with government support under United States Army Grant No. W911NF2120078. The government has certain rights in the invention.

### BACKGROUND

This disclosure relates generally to wearable assistance devices and systems, such as occupational wearable tools and exosuits, and methods of use.

Exos (e.g. rigid exoskeletons and soft exosuits) are wearable devices that assist, support, enable or augment human movement, posture, or physical activity through mechanical interaction with the body. Occupational exos are used to provide physical relief and assistance to workers in demanding jobs and to reduce musculoskeletal injury risks and fatigue. For instance, shoulder exos support the arms during overhead work and back exos assist lifting and bending. However, for users to accept and adopt exos, the devices must also be sufficiently comfortable, practical to use, and not interfere with other critical job tasks or movements.

Commercially available occupational exos do not fully meet the needs of some users. Barriers to exo adoption include incompatibility of the exo with required tasks or gear, poor fit, discomfort, time to don and doff, or the need for a device to be worn discreetly. If user needs are not met, an exo will not be used and thus will not contribute to a safer work environment. Workers who perform physical tasks sporadically, and at irregular, unpredictable intervals, are often not sure how to adopt or utilize exos. They are disinclined to carry around and keep track of extra tools. Occupational back exos commonly weigh between about 1-5 kg for passive exos and 3-7 kg for powered devices. Furthermore, exos take time to don and doff. Several commercially available exo manufacturers report donning times on the order of 30-60 seconds. While this is acceptable for most workers at the start of a shift, the time to don/doff during busy schedules can be a barrier to exo adoption for others. Furthermore, existing exos are not designed for prolonged sitting or driving because they contain rigid components on the back or around the hips which are incompatible with or uncomfortable during seated posture. Therefore, individuals who alternate between bending, lifting, and sitting tasks, or those in customer- or patient-facing jobs where it is important for devices to be discreet are generally not served by existing occupational exos.

### SUMMARY

The present disclosure envisions exosuits (also referred to as “exos”) that can be embedded into or worn under or over clothing to assist with lifting and bending, in order to make exo assistance accessible to more workers and/or military personnel. Currently, all occupational back exos on the commercial market are designed to be worn on top of a user's clothing, and they contain rigid components on the back or around the hips, which can be uncomfortable during sitting or restrict other movements, tasks or postures. Occupational exos have not yet been embedded or hybridized within regular clothing (e.g., uniforms, workwear).

Accordingly, the present disclosure provides, for example, a wearable assistance device that comprises a lower-body interface, an upper-body interface, and at least one elastic member mechanically coupling the upper-body interface to the lower-body interface. The elastic member comprises at least first and second elements associated with the lower-body and upper-body interfaces, respectively, the first and second elements being configured to provide an assistive force to the back of the wearer. A clutch mechanism is provided that is associated with the elastic member and is configured for selectively adjusting the assistive force provided by the first and second elements of the elastic member. A transmission member (also referred to as a “transmission”) extends from the clutch mechanism such that the clutch mechanism is spaced away from the second element of the elastic member, wherein the transmission member is configured to reorient or redirect forces relative to one or more elastic elements.

In some embodiments, the clutch mechanism is positioned at an outer lateral side of the lower-body interface; the clutch mechanism is located on the outer thigh of the wearer; the transmission member has a length that allows positioning of the clutch mechanism at an outer lateral side of the lower-body interface; and/or the transmission member is located on the lower-body interface.

In other embodiments, the first element of the elastic member comprises a rotary or power spring and the second element comprises an elastic band, fabric elastic, or similar spring-like or viscoelastic element; the transmission member comprises an outer sheath of a Bowden cable; the clutch mechanism is located between the first element of the elastic member and the transmission member; the clutch mechanism comprises a gear and pin or ratchet and pawl mechanism; the upper-body interface is a harness embedded into clothing; and/or the lower-body interface is one or more pant-integrated thigh sleeves and the upper-body interface is a shirt-integrated harness.

The present disclosure may also envision a wearable assistance device that comprises a lower-body interface, an upper-body interface, and at least one elastic member coupling the lower-body interface to the upper-body interface. The at least one elastic member has at least first and second elements associated with the lower-body and upper-body interfaces, respectively. The second element is configured to extend along the back of the wearer. A clutch mechanism is associated with the at least one elastic member and is coupled to the lower-body interface. The clutch mechanism is configured for selectively adjusting the assistive force provided by the first and second elements of the at least one elastic member. A transmission member is positioned between the first and second elements, and the transmission member has a length that extends between the clutch mechanism and the second element of the at least one elastic member such that the clutch mechanism is spaced away from the second element of the at least one elastic member. The clutch mechanism is located between the first element of the at least one elastic member and the transmission member. The transmission member is configured to redirect a downward force from the second element into a lateral force from the first element.

In certain embodiments, when the clutch mechanism is engaged and the wearer bends forward, a load path of the device goes from the lower-body interface through the clutch mechanism, through the transmission member, through the second element of the at least one elastic member, and through the upper-body interface, and bypasses the first element of the at least one elastic member; and/or when the clutch mechanism is disengaged and the wearer bends forward, a load path of the device goes from the lower-body interface through the clutch mechanism, through the first element of the at least one elastic member, through the transmission member, through the second element of the at least one elastic member, and through the upper-body interface.

In some embodiments, the first element of the at least one elastic member is a reset spring and the second element of the at least one elastic member is an elastic band; the reset spring is weaker (e.g., less stiff) than the elastic band; the lower-body interface is one or more pant-integrated thigh sleeves and the upper-body interface is a shirt-integrated harness; the at least one elastic member includes an intermediate element between the first and second elements thereof; the intermediate element is a rope segment; and/or the rope segment extends through the transmission member.

In one example, the wearable device comprises a wearable exosuit system with at least one or more elastic members and a transmission system to physically separate certain components. The transmission can used to separate the location of a clutch and/or a first element (e.g., reset spring) of the elastic member from the location of a second element (e.g., assistive spring) of the elastic member. One embodiment encompasses a back-assist exosuit with a clutch located over the outer side of each thigh. The upper-body interface and lower-body interface may be integrated into clothing such as uniforms or workwear, or they may be standalone accessories worn over or under clothing. The upper-body interface may be a harness, vest, backpack, shirt, or other body-worn apparel that is on the trunk or wraps over the shoulders. The lower-body interface may be leg sleeves worn over the thighs, knees, shanks, or feet, or other body-worn cuffs, pants, belts or wraps around the waist, buttocks, or legs. The upper-body or lower-body interface may include adjustment mechanisms, such as webbing and ladder lock buckles or other buckles, for tightening and loosening these interfaces around the user's body.

In some embodiments, the first element of the elastic member is affixed to a first or lower-body interface (e.g., thigh), and the second element is affixed to a second or upper-body interface (e.g., trunk). In some embodiments, the first and second elements of the elastic member are directly connected to their respective interface. In other embodiments, the first and second elements can be connected via an intermediate component, such as the clutch. The first and second elements of the elastic member may be connected to the body interface (directly or through intermediate components) at one end only or at multiple of their ends.

In some embodiments, the first element of the elastic member is positioned below the clutch and the second element of the elastic member is positioned above the clutch. That ordering may also be reversed. Or the entire system may be rotated such that the first element of the elastic member is positioned on one side of the clutch and the second element of the elastic member is positioned on a different side of the clutch.

An advantage of the wearable assistance device of the present disclosure over previous designs is that the transmission between the first and second elements of the elastic member enables the exosuit to be easily integrated into clothing. For example, the clutch can be more advantageously positioned on certain parts of the wearer's body to be more comfortable while sitting or to avoid interfering with other gear or personal protective equipment worn by the user. The transmission also means these two elements of the elastic member in the exosuit do not need to be co-linear, as in prior designs that involve multiple elastic elements. An additional advantage of the wearable assistance device of the present disclosure is that certain elements, such as the first elastic element (e.g. reset spring), can be made smaller and weaker if placed below the second elastic element, which enables the device to be lighter and more compact, and also to be more transparent to the user when they move.

The transmission can be flexible (e.g., flexible conduit, Bowden cable) or curved or conformal to the user's body or gear, to enable the exosuit to remain low-profile, lightweight, practical, and functional.

For purposes of this disclosure, the term “clutch” or phrase “clutch mechanism” may include any device that engages and disengages mechanical elements (e.g., elastic members) that bear or transmit force or mechanical power. A clutch mechanism may be unpowered such that it engages and disengages based on manual input or movement from the user. Alternatively a clutch mechanism may be powered such that a motor or other actuator with its own power supply is used to control engagement and disengagement, or to control the position of clutch engagement relative to one or more mechanical elements (e.g., elastic members), or to control the set point of an elastic member relative to the position of clutch engagement, thereby adjusting or setting tension of, for example, elastic member(s). The clutch mechanism may be used in combination with additional motors or actuators that provide force parallel, transverse or perpendicular to elastic members. For instance, the motors or other actuators may apply force in series with the elastic member(s), thus creating or controlling tension in the elastic member(s). The engaging and disengaging by the clutch mechanism of a mechanical element may be achieved by any form of clutch or brake, for example, a ratchet, dog clutch, cam clutch, friction clutch, overrunning clutch disc brake, drum brake, latch, or buckle.

One embodiment of the present disclosure is a back-assist exosuit, where the additional transmission system enables the reset spring and/or clutch to be positioned on the side of a user's thigh, rather than along the user's back, to improve comfort during sitting. In this embodiment, the second interface may be on the shoulders or trunk of the user, and the first interface may be on one or both thighs. In embodiments with two thigh interfaces, there may be one or more clutches to correspond with one or more elastic members.

Additional embodiments can be designed to assist other body joints, such as the neck, shoulder, elbow, foot, ankle, knee, hip, or thigh. For instance, in one embodiment assistance is provided about the hip but not the back by using a waist belt or similar attachment to the user's pelvis as the upper-body interface, rather than a shoulder harness. Or alternatively, a waist belt could be used as the lower-body interface with an upper-body interface on the trunk. In some embodiments, the upper-body interface and lower-body interface attach to body segments that are distal and proximal (or proximal and distal) to the body joints being assisted. Elastic elements may contain any amount of elasticity (e.g., could be viscoelastic). One or both elements of the elastic member could also be replaced with or put in series with any other type of actuator, such as a damper or electric motor. For example, the reset spring (first elastic element) could be replaced with an electric motor powered by a battery and controlled by a microcontroller. The motor could be inside or connected to the base component of the clutch housing.

This summary is not intended to identify essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1 are various views of an end-user (e.g. a soldier) wearing a wearable assistance device, according to an exemplary embodiment of the present disclosure, which comprises a standard-issue Army combat uniform integrated with quasi-passive back-assist capabilities to create a low-profile, uniform-integrated exosuit.

FIG. 2 are various views of an end-user similar to FIG. 1 in which fabric panels of the uniform are removed to show the main components of the uniform-integrated exosuit.

FIG. 3 are various views of the end-user similar to FIGS. 1 and 2 in which the shirt or upper part of the uniform is lifted to show the main components of the uniform-integrated exosuit.

FIG. 4 is a view of an exemplary lower body interface of the wearable assistance device of the present disclosure, positioned on the thighs with the clutch, a switch, and transmission systems.

FIG. 5 is an exploded view of an exemplary clutch of the wearable assistance device of the present disclosure that uses a spring-loaded pin and geared spool (sprocket) to engage and disengage.

FIG. 6 is a representative example showing the negligible differences in the reachable workspace for a user wearing a standard Army combat uniform (green) and the uniform-integrated exosuit (brown) of the present disclosure. The figure shows the convex hull of the full reachable workspace (left), a top-down view of the workspace (middle), and a side view of the workspace (right). These results demonstrate that assistance capabilities can be integrated into a standard uniform without restricting the user's natural range of motion.

FIG. 7 is a view inside the clutch with the spool partially extracted to show how the rope comes off the spool and is routed through the transmission member.

### DETAILED DESCRIPTION

The present disclosure generally relates to a wearable assistance device, such as aback-assist exosuit, that can be integrated or embedded into regular clothing in an effective and usable manner, to make musculoskeletal relief accessible to more workers. The device has added lifting assistance with little change to the form factor, weight, range of motion, or comfort of the clothing, such as a standard uniform. This is beneficial to workers who alternate between bending, lifting, and sitting (e.g., driving) tasks, or to those in customer- or patient-facing jobs where it is important for wearable technology to be discreet. Also, embedding an exo into or under clothing makes such physical assistance capabilities more readily available, and thus more accessible to workers for whom existing exos do not acceptably fit into their daily activities.

The wearable assistance device and system of the present disclosure can add exo assistance into or onto clothing while still satisfying other user/wearer needs related to comfort, practicality, and freedom of movement. As such, the wearable assistance device and system of the present disclosure can integrate the physical assistance and mode-switching capabilities of a standalone, accessory back exo into regular, daily clothing. In an example, the wearable assistance device can be a quasi-passive back exosuit integrated into workwear to provide convenient, on-demand assistance for people who perform strenuous lifting or bending at irregular intervals, or that need to sit intermittently (e.g., delivery drivers), or that require discreet assistance (e.g., nurses). The device can be comprised of, for example, a shirt-integrated harness to interface with the wearer's upper body, a pant-integrated thigh sleeves to interface with the lower body, elastic assistive bands that act as an artificial set of back and hip extensor muscles, and a clutch-switch system for engaging/disengaging assistance.

The wearable assistance device can be designed to maintain the familiar form and function of clothing. Also, the wearable assistance device incorporates one or more transmission members to position any protruding and rigid elements, such as the clutch mechanism, of the device in areas where people are already accustomed to carrying items, such as in or near pockets. In one example, the wearable assistance device does not have any or has minimal rigid protrusions on the back or back side of the body for comfort during seated tasks. The device is also configured to allow a natural range of motion while disengaged (e.g. the clutch is disengaged but the wearer is still wearing the device). And the device is designed to be comfortable during typical tasks, such as walking, bending, squatting, climbing, entering/exiting a vehicle, and sitting. In an embodiment, the design of the wearable assistance device takes into account a number of factors. For example, the wearable assistance device is designed to be donned and doffed like a standard uniform to ensure a familiar user experience. The device can also be configured to have a low-profile design (for example, defined as no components protruding more than 30 mm from the body) to ensure comfort and minimize interference, such as with Soldier gear and duties, especially while driving and sitting in chairs or vehicles with seatbacks. This low-profile design accounts for common items carried in pocket (such as that the sizes of items that Soldiers commonly carry), aims to place protruding or rigid elements of the device in areas where people are already accustomed to carrying items, such as in or near pockets, and avoids adding rigid protrusions on the back of the body for maximal comfort, such as during prolonged sitting.

The wearable assistance device may include physical assistance and mode-switching capabilities. The wearable assistance device provides back assistance torque during bending and lifting, which has been found to be sufficient to reduce back muscle activity and fatigue. In case studies, detailed more below, human subjects wearing the device received 15-27 Nm of back assistance torque during lifting. The wearable assistance device is also configured to be able to engage/disengage rapidly via a clutch mechanism, which is in less than 1 second, to provide on-demand lifting assistance. The wearable assistance device can incorporate one-handed mode switching (between engaged and disengaged modes) to allow for many real-life scenarios in which a user/wearer, such as a worker or soldier, may only have one hand available. Alternatively, the device could be designed to mode switch with two hands or no hands (handsfree), such as by using voice-activation or electronic sensors or microcontrollers to mode-switch.

The present disclosure can be applied to quasi-passive (mode-switching) exosuits that have a reset spring and assistive springs. A motor and/or sensors could optionally be included in certain embodiments. The wearable assistance devices and systems of the present disclosure can be used to create a novel exosuit that positions any rigid components away from undesirable locations on the body, using one or more transmission members, which can make the exosuit more practical for certain users. For instance, the present disclosure can be applied to uniquely embed an exosuit into clothing or uniforms.

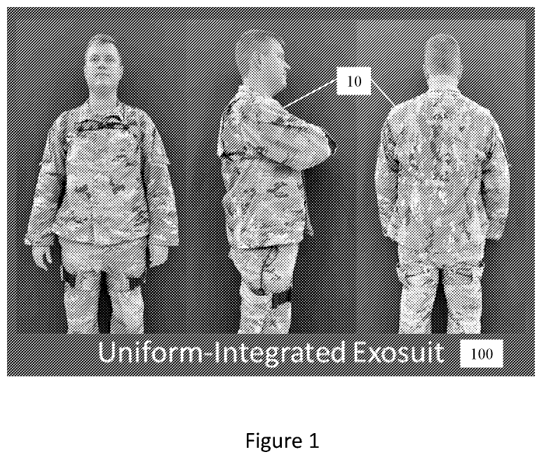

Referring to FIGS. 1-4, the wearable assistance device 100 can be an exosuit with physical assistance capabilities integrated into clothing, such as a workwear or military uniform 10, and comprises a lower-body interface 1 (e.g. one or more leg sleeves), an upper-body interface 2 (e.g. one or more shoulder harness), one or more elastic members which can each comprise first and second elastic elements 3a and 3c, a transmission member 4 (e.g. Bowden conduit or cable), and a clutch 5 (also referred to as “clutch mechanism”). The device 100 may also include a switch 7 and a switch transmission 7a (e.g. a Bowden cable) to control the clutch 5 on each thigh of the wearer.

The term “exosuit” refers broadly to a type of exo that is constructed, at least in part, from soft materials such as textiles and elastomers. An exosuit is also known as a soft exoskeleton or soft exo. An exosuit can be powered (e.g., motorized), passive, or quasi-passive. The exosuit can include high-tech, breathable fabric, padding, or slip-resistance features in the right places to comfortably conform to the wearer's body.

The term “body interface” refers to the component of the exosuit (e.g., a sleeve, strap, cuff) that physically touches, attaches to, or applies force to the wearer's body. The body interface may be positioned against the skin or inside or on top of the wearer's clothing. The clothing of the wearable device is designed to be taken on and off like normal clothing. In some embodiments, the wearable device is lightweight (e.g. adding less than 1.25 kg to a uniform), low-profile (i.e., no components that protrude more than 30 mm from the body), and comfortable during non-lifting tasks, and allows a user to retain natural range of motion.

The lower-body interface (e.g., thigh sleeves) 1 are configured to wrap around each leg. They may be comprised of soft goods (fabric, foam, and elastic materials). The thigh sleeves 1 can be attached to clothing, such at the back of pants, e.g. approximately 25 cm below the waistline, by any type of attachment, such as sewing. The thigh sleeves 1 can also be supported by a fabric loop (similar to a belt loop) that can be attached to, e.g. sewn, to the front of the pants. The upper-body interface or harness 2 can comprise shoulder straps and a flexible backplate covered in fabric. The harness 2 can be attached or embedded into clothing, e.g. uniform 10, in any manner, such as being sewed into clothing for the wearer's upper body, such as a shirt. The harness 2 can also include a chest strap for additional comfort of the wearer. In one example, a piece of camo fabric can be added onto the back to cover the harness 2 and elastic bands (second element of the elastic member). That serves to reduce snag risk and maintain the appearance of a standard uniform.

Each second elastic element 3c can be an elastic band or other elastic or viscoelastic element configured to provide an assistive force to the back of the wearer, for instance, to provide lifting assistance when a user bends forward. The elastic band or bands can stretch, thereby creating an extension torque about the lumbar spine that reduces strain on the back muscles and discs. The second elastic elements 3c connect the harness 2 to the thigh sleeves 1, via the clutch 5, and can run along the back of the wearer. During bending or lifting, when the clutch 5 is engaged, these elastic bands 3c stretch, creating an extension torque about the lumbar spine that reduces strain on the back muscles and discs, and an extension torque about the hips which further assists with lifting or standing upright.

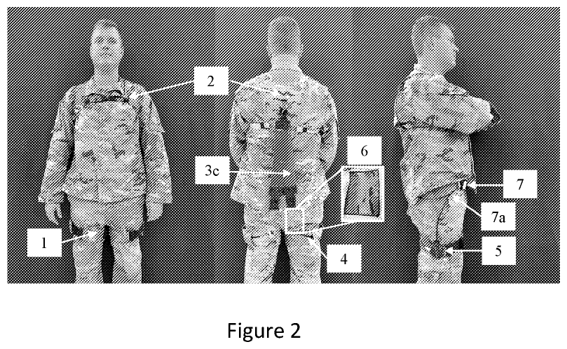

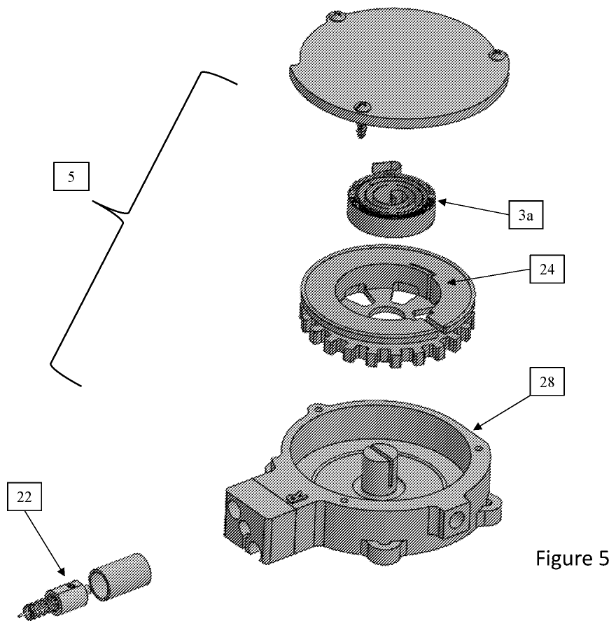

The clutch 5 can be attached (e.g. sewn) to the lower-body interface 1 at a convenient and comfortable location, e.g. to the lateral side of each thigh sleeve 1, to the front side of each thigh sleeve 1, around the waist, or elsewhere on the lower-body interface. In an example, the clutch 5 is attached to the lateral side of each thigh sleeve which can each be the lower-body interface 1. That clutch location avoids rigid or protruding components on the back or back side of the user, which can affect seated comfort. Thus, in this embodiment, the clutch 5 is not on the back of the user. A fabric cover can be added over the clutch 5 to reduce snag risk and maintain the appearance of a standard uniform. The clutch 5 can be designed to behave like a lockable keychain retractor. The clutch 5 can be a rotary type of clutch comprised of a geared spool 24 and spring-loaded pin 22, and can be located with the first elastic element 3a, which can be a rotary spring, as seen in FIG. 5. A rope or rope segment can be wrapped around the spool 24 and also connects to the elastic bands 3c, as seen in FIG. 2. The elastic element or spring 3a inside the spool 24 causes it to reel in the rope when the clutch 5 is disengaged and help keep the elastic member taut or lightly tensioned. When engaged, the pin 22 inserts into the gear teeth on the spool 24. That prevents rotation of the spool 24 and locks it in its current orientation.

Although the first elastic element 3a is shown inside of the housing of the clutch 5, the first elastic element 3a does not have to be inside or part of the clutch mechanism. For example, the elastic member 3a can run through a friction cam type clutch, such that the first elastic element 3a would be the portion of the elastic member on one side of the clutch and it would not need to be inside the housing of the clutch.

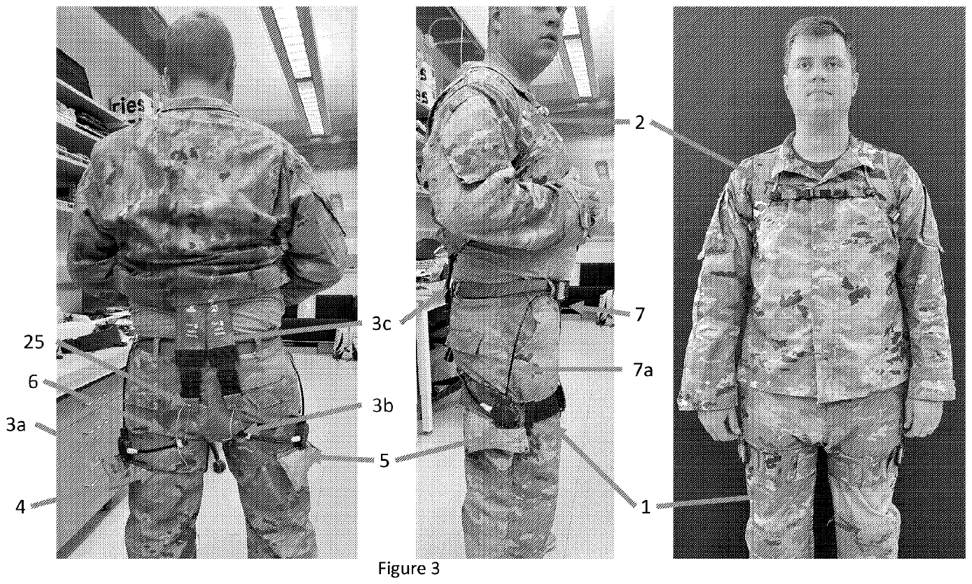

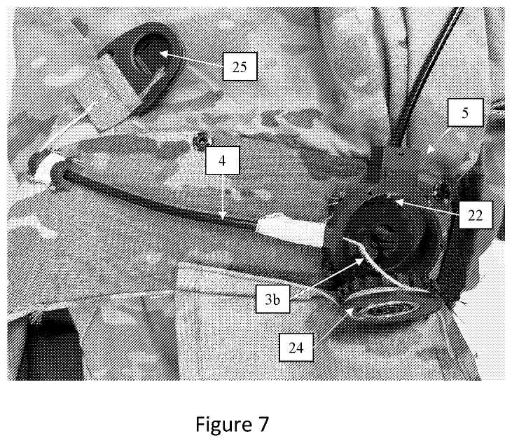

The transmission member 4 connects each clutch 5 to the second elastic element 3c (e.g. assistive elastic band in FIG. 3). The transmission member 4 extends from the clutch 5 at the lateral or outer side of the thigh sleeve 1 to the posterior or inner side of the thigh sleeve 1. The transmission member 4 can have a length that allows positioning of the clutch 5 at the outer lateral side of the lower-body interface. The transmission member 4, can be, for example, a Bowden conduit or other flexible or curved transmission system. One end of the transmission member 4 can include a segment of the rope 3b coming from the spool 24 of the clutch 5, as shown in FIG. 7. In an example, the transmission 4 can have an outer housing that extends from the clutch 5 to an end terminal affixed to the posterior side of the thigh sleeve and an inner portion of the transmission 4 is comprised of the rope coming from the spool. The transmission member 4 is configured to reorient or redirect forces relative to the one or more elastic elements. In one embodiment, the transmission member 4 redirects the downward force (axially along the back of the wearer) from the second element 3c into a lateral force from the first element 3a. That redirection of force is accomplished by the transmission member 4, which can be curved, that is attached to the lower-body interface 1 and through which runs the transmission rope 3b that connects the first and second elements 3a and 3c. This transmission rope 3b can serve as a third element of the one or more elastic members, and can be located between the first and second elastic elements, 3a and 3c. For ease of donning and doffing, a center release buckle 25 or any other kind of detachable connector can be provided connecting the rope 3b to the elastic band 3c, as shown in FIG. 3 and FIG. 7.

Each elastic member may comprise the first elastic element 3a (e.g. power spring in FIG. 5) and the second elastic element 3c (e.g. elastic bands in FIG. 2). The one or more elastic members and the first and second elements 3a and 3c thereof may be comprised of springs, elastic bands, bungee cords, webbing, straps, or any other component or material containing any amount of elasticity or viscoelasticity. There may also be more than two elements of each elastic member, such as an intermediate element (e.g. piece of strand or rope or webbing, like rope 3b), in between the first and second elements 3a and 3c. A motor or other actuator may also be placed in series with one or more of the elastic members to further control the force or displacement of the elastic member, for instance, to adjust different levels of assistance, adjust elastic spring set points, or switch between engagement (assistance) and disengagement (non-assistance) behaviors. A motor or other actuator may replace one or more elastic elements in the one or more elastic members.

The exosuit of the present disclosure is designed to be comfortable to sit in for long periods of time but can also provide assistance on demand when needed. An example use case would be for someone like a delivery driver who performs heavy lifting in between bouts of driving or sitting. In one embodiment, any rigid components of exosuit are removed from or minimized at the back of the uniform. Thigh sleeves 1 are built into the pants or worn on top of the pants and comprise the first or lower body interface. For simplicity, the configuration of a single thigh sleeve is described, but in practice, this is duplicated on the other leg. A shoulder harness 2 can be similar to backpack straps and can comprise the upper body interface. Each elastic member can comprise a reset spring (biased or configured to retract or pull in rope 3b) as the first element 3a, an elastic band as the second element 3c (also referred to herein as an assistive band), and a strand or rope as the intermediate element 3b that connects that reset spring 3a and elastic band 3c. The clutch 5 can be positioned between the reset spring 3a and the rope 3b.

The reset spring 3a and the clutch 5 can be positioned on the pants thigh, for instance, on the lateral side or front. The transmission 4 directs the rope 3b from going downward at the back of the leg (where it connects to the assistive band) to going laterally on the side of the leg (where it connects with the reset spring 3a). That redirection of forces can be an angular redirection (reorientation) or a change in the line of action of the force. That allows the clutch 5 and reset spring (first elastic element 3a) to be non-collinear with the elastic band (second elastic element 3c), such as when a person is standing upright. That redirection of force allows the exosuit components to be placed in the most advantageous locations on the body, such as moving rigid components off the back of the user to improve seated comfort.

In an embodiment, when the clutch 5 is engaged (or locked), and the user/wearer bends forward, the load path goes from the lower-body interface 1 through the clutch 5, through the transmission member 4 (e.g. Bowden cable outer housing), through rope 3b, elastic band element 3c, and upper-body interface 2. In other words, the reset spring (first element 3a of the elastic member) is bypassed. That allows the assistive elastic element 3c to stretch or deform, which provides the assistance during bending and lifting by providing a force parallel to the user's back muscles and/or a torque about the spine or hips of the user. As such, instead of all of the force going through the user's lower back musculature, some of the force needed to lift, lower or bend forward is now provided by the wearable assistance device 100.

In an embodiment, when the clutch 5 is disengaged, and the user/wearer bends forward, there is a different load path: the load path goes through all the aforementioned elements and also through the reset spring 3a. The reset spring 3a can be a weaker spring (e.g., lower stiffness) than the elastic band 3c, which allows freedom of movement with negligible force or resistance generated by the deformation of the reset spring 3a. The reset spring 3a can provide a smaller amount of tension to prevent too much slack or prevent unwanted snag risks from the exosuit. For this embodiment, the reset spring 3a can be affixed at both of its ends, that is, at the lower-body interface 1 and at the clutch 5, respectively. Alternatively, in some other embodiments, the reset spring 3a can be replaced by another element, such as a webbing, and may only be affixed at one end (e.g., at the clutch end), such that it slides or displaces when the clutch 5 is disengaged and the user bends forward. In one exemplary embodiment, the clutch 5 includes a friction cam and base, the first element 3b slides between the friction cam and base when the clutch is disengaged, and the first element 3b can be fixated by clamping and friction forces between the cam and base when the clutch is engaged.

The first elastic element 3a is attached to the lower-body interface 1, such as via the clutch 5. For example, the first elastic element or reset spring 3a can be connected on one end to the lower-body interface 1 via a base component 28 (FIG. 5) of the housing of the clutch 5. The first elastic element 3a is configured to serve as a reset spring to remove slack from the wearable system. The first elastic element 3a may be less stiff than the second elastic element 3c. In other embodiments, the elastic elements 3a and 3c are the same stiffness. In yet other embodiments, the first element 3a can be stiffer than the second element 3c of the elastic member, such as when the reset spring 3a is only affixed at one end. In such embodiments, the user may manually exert force or pull on element 3a to adjust the tautness or setpoint of the elastic member.

The second elastic element 3c is attached to the upper-body interface 2. When stretched, the second elastic element 3c generates a force that assists or resists the wearer's movement. The rope 3b connects the first elastic element 3a to the second elastic element 3c, and travels through the transmission member 4, which is affixed on at least one end to the lower-body interface 1. The transmission member 4 may be curved or conformal to the wearer's body. The transmission members 4 may be comprised of a Bowden cable, flexible conduit system, pulley, track system, or other system that serves to reorient or redirect forces relative to one or more elastic elements. The transmission member 4 may span zero, one, or multiple body segments.

Each clutch 5 can be located between the first and second elastic elements 3a and 3c. The clutch 5 is configured to change the load path between the upper and lower-body interfaces through the one or more of the elastic members, when the clutch 5 is engaged, i.e. changes from disengaged to engaged. For instance, when the clutch 5 is disengaged (also referred to as “disengaged mode”), the load path may go through the first and second elastic elements 3a and 3c, but when the clutch 5 is engaged (also referred to as “engaged mode”), it may change the load path to go through only the second elastic element 3c. In one embodiment, which would allow for unrestricted user movement when the clutch 5 is disengaged due to minimal force generated by displacement of a first elastic element. And it would allow for larger assistive forces to be generated by the displacement of the second elastic element 3c when the clutch 5 is engaged.

In an exemplary embodiment, the first elastic element 3a comprises a rotary spring or power spring, similar to what is found in a key chain retractor or retractable dog leash. The second elastic element 3c comprises an elastic band, fabric elastic, or similar spring-like element. The transmission member 4 comprises the outer sheath of a Bowden cable that is configured to redirect the downward (axially along back) force from the second elastic element 3c into a lateral force from the first elastic element 3a. The transmission can be curved and affixed at both ends to the lower-body interface. The rope 3b slides through the outer sheath of the transmission 4 and is redirected along the curved path. The clutch 5 is located between the first elastic element 3a and the transmission 4.

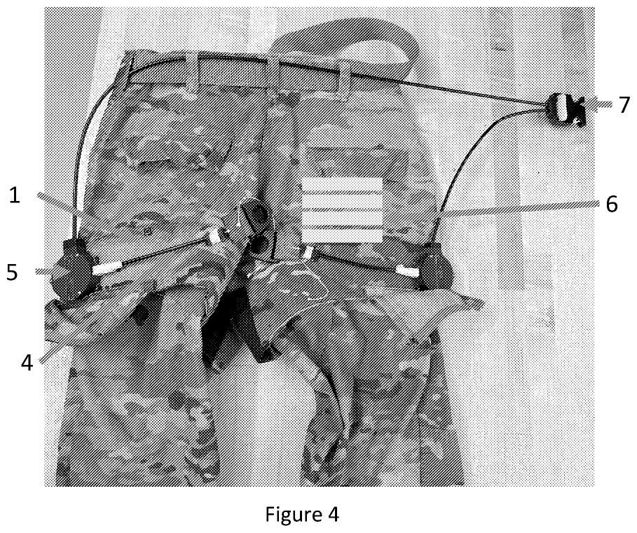

FIG. 4 illustrates an exemplary embodiment of the lower-body interface 1 of the wearable assistive device 100 showing the lower-body interface 1 (e.g. thigh sleeves), the transmission member 4, clutches 5, a rear protector 6 embedded inside of the pants of the uniform (yellow rectangles indicate where slats are sewn inside the pants), and the switch 7 (one manual switch controls engagement and disengagement of both clutches 5, one on each thigh). The rear protector 6 is configured to prevent high pressure concentrations on the user's back, buttocks or legs from the rope 3b. In one embodiment, that rear protector 6 can be comprised of a series of horizontal slats of thin plastic (e.g., approximately 0.075 inches thick), or made of other thin materials or shapes. The slats allow the protector to flex in a way that conforms to the buttocks when a user bends forward. The slats are thinner than other items commonly carried in a person's back pocket, such as a wallet. The slats also distribute elastic member forces over a larger surface area of the user's body to enhance comfort of the user. That, for instance, can reduce pressure concentrations from a taut rope 3b against the buttocks or legs. The rear protector 6 is not required, for instance, if the rope 3b is replaced with wider webbing and the transmission member 4 is shaped to allow this webbing to slide through. The switch transmission 7a connects each clutch 5 to the switch 7 (e.g. a single buckle-style switch) that allows the user to manually engage or disengage assistance as desired. The switch 7 can be affixed to a waist belt or other parts of the body and controls the pin mechanism 22 of the clutch 5 to lock or unlock the clutch 5. The switch can be manual or powered; for instance, it may consist of a motor or solenoid. Embodiments may contain one or more switches. In one embodiment, a single switch 7 controls all clutches 5. In other embodiments, there can be one switch 7 per clutch 5.

FIG. 5 is an exploded view of an exemplary clutch 5 according to the present disclosure that uses a pin and geared spool or sprocket 22 to engage and disengage the clutch. When the clutch 5 is engaged, the pin inserts into the gear teeth, which locks it in place and prevents it from rotating. When the clutch 5 is disengaged, the pin is removed from the sprocket/gear, such that it can rotate. In that rotary configuration, the first elastic element 3a is a power spring located inside the spool housing. In the disengaged mode of the clutch 5, the force (load path) of the elastic member goes through the first elastic element 3a. Whereas, in engaged mode of the clutch 5, the first elastic element 3a is bypassed. Instead, the load path goes from the upper-body interface (shoulder harness) through the second elastic element 3c, rope 3b, and clutch 5 to the lower-body interface 1 (thigh sleeves).

The present disclosure also relates to a method for engaging or disengaging the clutch 5. For instance, a switch, e.g. switch 7, is included to engage and disengage the clutch 5. The switch may comprise any kind of mechanism for turning a device on/off or for changing device modes, such as a button, toggle, buckle-style switch, flip tab or lever. The switch 7 may be located at a distance away from each clutch 5 and can control the clutch 5 via a mechanical transmission or switch transmission 7a (e.g., Bowden cable) or an electrical signal, such as transmitted wirelessly or via a cable. Alternatively, the switch 7 may be located on the clutch 5. In an example, the clutch-switch transmission, connects both clutches to a single buckle-style switch that allows the user to manually engage or disengage assistance as desired (FIG. 2). The switch can be affixed to the waist belt and controls the pin to lock or unlock the clutch. Neither the switch nor clutch requires a motor or battery, which avoids the need for recharging. Though in some embodiments, one or more batteries or motors or other actuators may be used. In an aspect, flexible plastic strips can be integrated into the seat of the pants to prevent discomfort from the rope running over the user's buttocks (, as seen in FIG. 2).

However, for some embodiments, the switch 7 is not necessary. For instance, some clutches leverage the user's body weight to engage the clutch when the foot is on the ground and to disengage the clutch when the foot is swinging through the air during locomotion. Also, there are various alternative ways to engage the clutch with different types of switches, buttons, voice-activation systems, wearable sensors, or similar user input mechanisms, or with different automatic mode-switching systems that may use body weight or sensors, microprocessors, and algorithms that toggle the clutch between modes without a user manually switching modes or providing a trigger input.

Additional embodiments of the wearable system can be designed to assist other body joints, such as the neck, shoulder, elbow, foot, ankle, knee, or thigh. Different elastic or viscoelastic elements can be used, such as linear and rotational springs and different materials for the springs (e.g., elastomer, fabric). Different methods of clutching and mode-switching can also be used. The transmission system could be implemented in various ways including but not limited to Bowden cables, flexible conduits, channels, or pulleys. The transmission system can cross joints. There may be one or more transmissions per clutch. For example, in an elbow flexion exosuit, the reset spring can be on the user's torso, the transmission can cross the shoulder and connect to the humerus, and the assistive spring can cross the elbow. Or in an ankle exosuit the transmission system can enable the reset spring and/or clutch to be positioned on the top or side of a user's shoe, rather than under the shoe, to improve device comfort, functionality, and/or reliability.

### Case Studies

Two human subject feasibility studies were performed using a prototype of the wearable assistance device of the present disclosure: a lab-based cases series to evaluate each design requirement (N=5) and a field-based case study on a Soldier during operationally relevant tasks (N=1).

In the lab study, the weight and form factor of the prototype was measured, and then participants performed a variety of movement tasks after a brief fitting, training, and acclimation period. Tasks included donning and doffing the prototype, sitting for three minutes (on a 45 cm tall chair with a seatback, no armrests, and no padding), performing sit-to-stand transitions, walking for 50 m, ascending/descending two flights of stairs, kneeling bilaterally and standing up, lunging, squatting, climbing over a 76 cm tall table, twisting their trunk left and right, and lifting and lowering a 10 kg box from the floor. Participants also performed omni-directional reaching tasks to evaluate their range of motion, and they completed surveys to assess comfort and range of motion.

In the field study, a case study was completed on a U.S. Army Soldier whose duties involved prolonged driving and intermittent heavy lifting. That Soldier's Military Occupational Specialty (MOS) was 88M (Motor Transport Operator). 88M Soldiers operate vehicles to transport cargo and troops and assist with loading and unloading of the vehicle. In the field artillery, that job can involve loading and unloading boxes weighing up to 55 kg. The Soldier was fitted with a prototype of the wearable assistance device of the present disclosure, and he was given 5 minutes to use and acclimate to it. The Soldier then completed a series of operationally relevant work tasks, such as loading a 55 kg artillery round box onto the back of a Heavy Expanded Mobility Tactical Truck (HEMTT), lifting and lowering the box multiple times, climbing up and down from the truck bed, climbing into and out of the vehicle cab, sitting in the driver's seat, and walking. Following those tasks, the Soldier completed a survey to assess their self-reported satisfaction with the prototype.

The results of the studies on the prototype were as follows.

Donning and Doffing. The uniform-integrated exosuit prototype was donned and doffed in a similar manner as a standard uniform, with a few brief additional steps. Doffing involves the following steps: (i) loosening the shoulder harness by pulling up on two ladder lock buckles, one on each shoulder, which is like loosening a standard backpack, (ii) loosening the thigh sleeves by pulling forward on two ladder lock buckles, one on each leg, (iii) disconnecting the elastic bands from the thighs using a center release buckle, (iv) taking off the pants and shirt as normal. For donning, this sequence was simply reversed: the center release buckle was connected, and webbing straps were pulled by the user to tighten ladder lock buckles on the shoulders and thighs. For both donning and doffing, the additional steps (i)-(iii) could be done by the wearer without assistance from another person. One participant performing steps (i)-(iii) took 15 seconds during donning and 10 seconds during doffing to complete, which is acceptable and sufficiently similar to normal clothing.

Weight and Profile. The prototype was 0.8 kg heavier than the standard uniform (2.3 kg vs. 1.5 kg). All exosuit protrusions were less than 30 mm. The maximum protrusions were from the buckle switch (25 mm) located on the waist belt and from each clutch (24 mm) located on the lateral thighs.

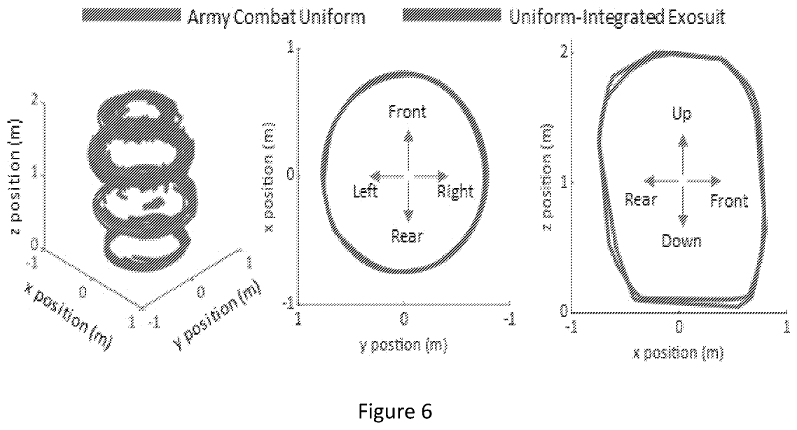

Range of Motion. The prototype did not restrict the reachable workspace of participants. The percent change in reachable workspace volume ranged from 0.9% (0.04 m³) smaller to 6.8% (0.22 m³) larger when wearing the prototype, as compared to the standard uniform. On average, the workspace was 1.6% (0.05 m³) larger when wearing the prototype than the standard uniform. Data from one representative participant are shown in FIG. 6. In the survey, 49 of 50 responses (10 tasks×5 participants) indicated that users did not feel any restriction in range of motion while wearing the prototype.

Comfort. Participants were generally comfortable in the prototype. Participants reported no discomfort for 45 of 50 survey responses (10 tasks×5 participants). There was no reported discomfort during sit-to-stand, walking, lunging, squatting, climbing over a table, or twisting the trunk.

Assistance. The exosuit provided greater than 15 Nm of back extension assistance during lifting for all participants. Across the five participants in the lab study, the average assistance (lumbar extension torque provided by the prototype) during lifting was 22±4 Nm and ranged from 18-27 Nm (assuming a moment arm of 12 cm). For moment arms of 8-15 cm, the mean peak torque was estimated to be 15-28 Nm.

Mode-switching. All participants were able to engage and disengage the switch one-handed. The average time to switch between modes was 0.58=0.06 seconds.

The Soldier survey indicated that he was “Very Satisfied” with the Weight, Comfort, and Range of Motion of the prototype during their work tasks and that he was “Slightly Satisfied” with the Lifting Assistance. The Soldier reported that he would be “Very Likely” to wear the wearable assistance device of the present disclosure.

This demonstrates that physical assistance and quasi-passive mode-switching capabilities of a back-assist exosuit can be integrated into standard workwear—in this case a standard-issue U.S. Army uniform—in an effective and usable way with little change to the form factor, weight, range of motion, or comfort of the clothing. This integration (i.e. uniform-integrated exosuit) of the present disclosure is a substantial and novel redesign of existing back exosuits, including moving the clutch mechanism from the back to the legs. This uniform-integrated exosuit meets or exceeds all design requirements while being sufficiently clothing-like in form-factor, weight, donning, doffing, comfort, and range of motion. However, unlike clothing, this uniform-integrated exosuit generated 22 Nm of assistive torque, on average, about the low back during bending and lifting. Furthermore, this magnitude of back assistance can reduce back muscle activity, fatigue, and injury risk. Quick, one-handed mode-switching is also provided which improves usability and user acceptance during real-world work.

It will be apparent to those skilled in the art having the benefit of the teachings presented in the foregoing descriptions and the associated drawings that modifications, combinations, sub-combinations, and variations can be made without departing from the spirit or scope of this disclosure. Likewise, the various examples described may be used individually or in combination with other examples. Those skilled in the art will appreciate various combinations of examples not specifically described or illustrated herein that are still within the scope of this disclosure. In this respect, it is to be understood that the disclosure is not limited to the specific examples set forth and the examples of the disclosure are intended to be illustrative, not limiting.

As used in this specification and the appended claims, the singular forms “a”, “an” and “the” include plural referents, unless the context clearly dictates otherwise. Similarly, the adjective “another,” when used to introduce an element, is intended to mean one or more elements. The terms “comprising,” “including,” “having” and similar terms are intended to be inclusive such that there may be additional elements other than the listed elements.

Additionally, where a method described above or a method claim below does not explicitly require an order to be followed by its steps or an order is otherwise not required based on the description or claim language, it is not intended that any particular order be inferred. Likewise, where a method claim below does not explicitly recite a step mentioned in the description above, it should not be assumed that the step is required by the claim.

It is noted that the description and claims may use geometric or relational terms, such as right, left, above, below, upper, lower, top, bottom, linear, arcuate, elongated, parallel, perpendicular, etc. Those terms are not intended to limit the disclosure and, in general, are used for convenience to facilitate the description based on the examples shown in the figures. In addition, the geometric or relational terms may not be exact.

What is claimed is:

1. A wearable assistance device, comprising:
a lower-body interface and an upper-body interface;
at least one elastic member coupling the upper-body interface to the lower-body interface, the at least one elastic member comprising at least first and second elements associated with the lower-body and upper-body interfaces, respectively, the first and second elements being configured to provide an assistive force to the back of the wearer;
a clutch mechanism associated with the elastic member, the clutch mechanism being configured for selectively adjusting the assistive force provided by the first and second elements of the at least one elastic member; and
a transmission member being positioned between the first and second elements, and extends from the clutch mechanism such that the clutch mechanism is spaced away from the second element of the elastic member,
wherein the transmission member is configured to redirect forces between the first and second elements.

2. The device of claim 1, wherein the clutch mechanism is positioned at an outer lateral side of the lower-body interface.

3. The device of claim 1, wherein the transmission member has a length that allows positioning of the clutch mechanism at an outer lateral side of the lower-body interface.

4. The device of claim 3, wherein the clutch mechanism is located on the outer thigh of the wearer.

5. The device of claim 1, wherein the transmission member is located on the lower-body interface.

6. The device of claim 1, wherein the first element of the elastic member comprises a rotary or power spring and the second element comprises an elastic band, fabric elastic, or spring-like element.

7. The device of claim 1, wherein the transmission member comprises an outer sheath of a Bowden cable.

8. The device of claim 1, wherein the clutch mechanism is located between the first element of the elastic member and the transmission member.

9. The device of claim 1, wherein the clutch mechanism comprises a gear and pin mechanism.

10. The device of claim 1, wherein the upper-body interface is a harness embedded into clothing.

11. The device of claim 1, wherein the lower-body interface is one or more pant-integrated thigh sleeves and the upper-body interface is a shirt-integrated harness.

12. A wearable assistance device, comprising:
a lower-body interface and an upper-body interface;
at least one elastic member coupling the lower-body interface to the upper-body interface, the at least one elastic member comprising at least first and second elements associated with the lower-body and upper-body interfaces, respectively, the second element being configured to extend along the back of the wearer;
a clutch mechanism associated with the at least one elastic member and coupled to the lower-body interface, the clutch mechanism being configured for selectively adjusting the assistive force provided by the first and second elements of the at least one elastic member; and
a transmission member being positioned between the first and second elements, and the transmission member having a length that extends between the clutch mechanism and the second element of the at least one elastic member such that the clutch mechanism is spaced away from the second element of the at least one elastic member,
wherein the clutch mechanism is located between the first element of the at least one elastic member and the transmission member, and
wherein the transmission member is configured to redirect a downward force from the second element into a lateral force from the first element.

13. The device of claim 12, wherein when the clutch mechanism is engaged and the wearer bends forward, a load path of the device goes from the lower-body interface through the clutch mechanism, through the transmission member, through the second element of the at least one elastic member, and through the upper-body interface, and bypasses the first element of the at least one elastic member.

14. The device of claim 12, wherein when the clutch mechanism is disengaged and the wearer bends forward, a load path of the device goes from the lower-body interface through the clutch mechanism, through the first element of the at least one elastic member, through the transmission member, through the second element of the at least one elastic member, and through the upper-body interface.

15. The device of claim 12, wherein the first element of the at least one elastic member is a reset spring and the second element of the at least one elastic member is an elastic band.

16. The device of claim 15, wherein the reset spring is weaker than the elastic band.

17. The device of claim 12, wherein the lower-body interface is one or more pant-integrated thigh sleeves and the upper-body interface is a shirt-integrated harness

18. The device of claim 12, wherein the at least one elastic member includes an intermediate element between the first and second elements thereof.

19. The device of claim 18, wherein the intermediate element is a rope segment.

20. The device of claim 19, wherein the rope segment extends through the transmission member.
